(19) United States
(12) Patent Application Publication
(10) Pub. No.: US 2008/0216835 A1
(43) Pub. Date: Sep. 11, 2008

(54) STANDALONE CPAP DEVICE AND METHOD OF USING

(75) Inventors: William J. McGINNIS, Cincinnati, OH (US); Scott A. Metrick, Lake Forest, IL (US)

Correspondence Address: PATENT ADMINISTRATOR;KATTEN MUCHIN ROSENMAN LLP, 1025 THOMAS JEFFERSON STREET, N.W., EAST LOBBY: SUITE 700, WASHINGTON, DC, 20007-5201, US

(73) Assignee: NeuroPhysiological Concepts LLC, Glenview, IL (US)

(21) Appl. No.: 11/928,436

(22) Filed: Oct. 30, 2007

Related U.S. Application Data
(63) Continuation-in-part of application No. 11/683,633, filed on Mar. 8, 2007.

(51) Int. Cl. A61M 16/00

(52) U.S. Cl. 128/204.23

(57) ABSTRACT

A continuous positive airway pressure (CPAP) device is provided. The CPAP device includes the feature that the blower is mounted directly to the mouthpiece or the nose mask, thus eliminating the need for an air tube to convey the pressurized air from the blower to the user's mouth or nose. The CPAP device may operate in continuous mode, bi-phase mode, or automatic mode. The CPAP device may include a capability to regulate pressure, temperature, and/or relative humidity of the pressurized air. The CPAP device may collect and store user respiration data, possibly including recordation of snoring noises.

### CROSS-REFERENCE TO RELATED APPLICATIONS

This patent application is a continuation-in-part of U.S. application Ser. No. 11/683,633, filed on Mar. 8, 2007, the contents of which are incorporated herein in their entirety.

### BACKGROUND OF THE INVENTION

1. Field of the Invention

The present invention relates to treatment and/or diagnosis of partial or complete upper airway occlusion, and more particularly to a stand-alone continuous positive airway pressure (CPAP) device and an associated method of using a CPAP device to treat and/or diagnose sleep apnea disorders.

2. Description of the Related Art

Sleep apnea is a syndrome in which a person stops breathing during sleep. When the airflow ceases for more than 10 seconds at a time, the syndrome is referred to as “apnea”. Apneas can lead to decreased blood oxygenation, and can often disrupt sleep. Apneas can be categorized as either central apneas, in which there is no respiratory effort, or as obstructive apneas, in which there is some respiratory effort. In some central apneas and all obstructive apneas, the airway becomes completely closed. This closure usually occurs at the level of the tongue or soft palate. Finally, the airway may also be only partially obstructed, which can also lead to decreased ventilation (hypoapnea) and decreased blood oxygenation, as well as disturbed sleep.

A conventional treatment of sleep apnea is the administration of continuous positive airway pressure (CPAP). It is believed that the CPAP treatment acts as a pneumatic splint of the airway by the provision of a positive pressure. The ancillary air is often supplied to the airway by a motor-driven blower whose outlet passes air, via an air delivery hose, to a nose (and/or mouth) mask sealingly engaged to a patient's face. An exhaust port is often provided somewhere along the deliver hose proximate to the mask. The mask is often either a nose and/or face mask or nasal prongs, pillows, or cannulae.

Sometimes the CPAP device warms the air through the compression in the fan, and the relative humidity of the air is consequently reduced. This dry air created by the CPAP device can sometimes lead to irritation of the mucous membranes of the respiratory passages by desiccation.

While presently known CPAP techniques fulfill many of their respective objectives and requirements, there are no known CPAP techniques or devices that have the interconnected components of a mouthpiece, a housing, an airway, a blower, a cover, a control circuit, and an on/off switch for treating and/or diagnosing sleep apnea disorders. This combination of interconnected elements could be specifically designed to match a user's particular individual needs, thus making it possible to provide a stand-alone means for treating and/or diagnosing sleep apnea disorders in a convenient manner.

Therefore, a need exists for a new and improved CPAP device and an associated method of using the new and improved CPAP device for treating and/or diagnosing sleep apnea. In this respect, the CPAP device according to the present invention substantially departs from the conventional concepts and designs of the prior art, and in so doing, provides an apparatus primarily developed for the purpose of providing a convenient means for treating and/or diagnosing sleep apnea, in the form of a stand-alone CPAP device.

### SUMMARY OF THE INVENTION

The present CPAP device and method of using a CPAP device, according to principles of the present invention, overcome a number of the shortcomings of the prior art by providing a novel CPAP device for use in treating and/or diagnosing sleep apnea disorders. In view of the foregoing disadvantages inherent in known conventional CPAP devices, the present invention provides an improved CPAP device which is not anticipated, rendered obvious, suggested, or implied by the prior art, either alone or in any combination thereof. It is therefore an object of the present invention to provide a new and improved CPAP device that has many of the advantages of conventional CPAP devices and that minimizes a number of the aforementioned disadvantages. It is another object of the present invention to provide a new and improved CPAP device that may be easily and efficiently manufactured and marketed. It is yet another object of the present invention to provide a new and improved CPAP device that has a low cost of manufacture with regard to both materials and labor, and which accordingly is susceptible of low prices of sale to the consuming public, thereby providing a CPAP device that is economically available to the general public.

Accordingly, in one aspect, the present invention provides a continuous positive airway pressure (CPAP) system. The CPAP system comprises a mouthpiece configured for being positioned partially within a mouth of a user; a housing mounted to the mouthpiece, the housing including an airway; and a blower coupled directly to the housing, the blower being in fluid communication with the airway. The blower is configured to provide pressurized air to the mouth of the user for the prevention of respiration stoppages. The CPAP system may further include a humidifier chamber coupled directly to the blower and to the housing, the humidifier chamber being in fluid communication with the airway, and a cartridge coupled directly to the humidifier chamber.

The CPAP system may further include a control circuit, the control circuit being operatively coupled to the blower, and at least one detector, the at least one detector being operatively coupled to the control circuit. The control circuit may be configured to control a mode of operation selected from the group consisting of a continuous mode, a bi-phase mode, and an automatic mode. The at least one detector may include an air pressure detector. The control circuit may be further configured to control a pressure level of the pressurized air. The at least one detector may include a temperature detector. The control circuit may be further configured to control a temperature of the pressurized air. The at least one detector may include a humidity detector. The control circuit may be further configured to control a relative humidity of the pressurized air.

The CPAP system may further include a flash memory, the flash memory being operatively coupled to the control circuit and the at least one detector. The flash memory may be configured to store data detected by the at least one detector. The CPAP system may further include a microphone, the microphone being operatively coupled to the control circuit, and a speaker, the speaker being operatively coupled to the control circuit. The microphone may be configured to detect snoring noises emitted by the user. The speaker may be configured to output audible sound. The audible sound may be selected from the group consisting of a noise cancellation function with respect to the detected snoring noises, an alarm sound for awakening the user from sleeping, and a musical melody.

In another aspect, the invention provides a continuous positive airway pressure (CPAP) system. The system comprises a nose mask configured for being sealingly positioned over a nose of a user; a housing mounted to the nose mask, the housing including an airway; and a blower coupled directly to the housing, the blower being in fluid communication with the airway. The blower is configured to provide pressurized air to a nasal cavity of the user for the prevention of respiration stoppages. The CPAP system may further include a humidifier chamber coupled directly to the blower and to the housing, the humidifier chamber being in fluid communication with the airway, and a cartridge coupled directly to the humidifier chamber.

The CPAP system may further include a control circuit, the control circuit being operatively coupled to the blower, and at least one detector, the at least one detector being operatively coupled to the control circuit. The control circuit may be configured to control a mode of operation selected from the group consisting of a continuous mode, a bi-phase mode, and an automatic mode. The at least one detector may include an air pressure detector. The control circuit may be further configured to control a pressure level of the pressurized air. The at least one detector may include a temperature detector. The control circuit may be further configured to control a temperature of the pressurized air. The at least one detector may include a humidity detector. The control circuit may be further configured to control a relative humidity of the pressurized air.

The CPAP system may further include a flash memory, the flash memory being operatively coupled to the control circuit and the at least one detector. The flash memory may be configured to store data detected by the at least one detector. The CPAP system may further include a microphone, the microphone being operatively coupled to the control circuit, and a speaker, the speaker being operatively coupled to the control circuit. The microphone may be configured to detect snoring noises emitted by the user. The speaker may be configured to output audible sound. The audible sound may be selected from the group consisting of a noise cancellation function with respect to the detected snoring noises, an alarm sound for awakening the user from sleeping, and a musical melody.

In yet another aspect, the invention provides a method for treatment of sleep apnea in a user. The method comprises the step of providing pressurized air to a mouth of the user by using a continuous positive airway pressure (CPAP) device. The CPAP device includes a mouthpiece configured for being positioned partially within the mouth of the user, a housing mounted to the mouthpiece and having an airway, and a blower coupled directly to the housing and in fluid communication with the airway.

The method may further include the steps of selecting a mode of operation of the CPAP device from the group consisting of a continuous mode, a bi-phase mode, and an automatic mode; and regulating a pressure of the pressurized air based on the selected mode of operation. The method may further include the step of regulating a relative humidity of the pressurized air. The method may further include the step of regulating a temperature of the pressurized air. The method may further include the steps of detecting data relating to respiration by the user and recording the detected data. The step of detecting data relating to respiration may include detecting snoring noises emitted by the user. The method may further include the step of outputting audible sound. The audible sound may be selected from the group consisting of a noise cancellation function in respect of the detected snoring noises, an alarm sound for awakening the user from sleeping, and a musical melody.

In still another aspect, the invention provides a method for treatment of sleep apnea in a user. The method comprises the step of providing pressurized air to a nasal cavity of the user by using a continuous positive airway pressure (CPAP) device. The CPAP device includes a nose mask configured for being sealingly positioned over a nose of the user, a housing mounted to the nose mask and having an airway, and a blower coupled directly to the housing and in fluid communication with the airway.

The method may further include the steps of selecting a mode of operation of the CPAP device from the group consisting of a continuous mode, a bi-phase mode, and an automatic mode; and regulating a pressure of the pressurized air based on the selected mode of operation. The method may further include the step of regulating a relative humidity of the pressurized air. The method may further include the step of regulating a temperature of the pressurized air. The method may further include the steps of detecting data relating to respiration by the user and recording the detected data. The step of detecting data relating to respiration may include detecting snoring noises emitted by the user. The method may further include the step of outputting audible sound. The audible sound may be selected from the group consisting of a noise cancellation function in respect of the detected snoring noises, an alarm sound for awakening the user from sleeping, and a musical melody.

### BRIEF DESCRIPTION OF THE DRAWINGS

FIG. 1 is a perspective view of a CPAP device constructed according to a preferred embodiment of the invention.

FIG. 2 is an exploded view of a CPAP device, according to a preferred embodiment of the invention.

FIG. 3 is a frontal view of a CPAP device, according to a preferred embodiment of the invention.

FIG. 4 is a side view of a user wearing a CPAP device having a mask that provides air directly into the nasal cavity, according to a preferred embodiment of the invention.

FIG. 5 is a front view of a user wearing the CPAP device of FIG. 4.

FIG. 6 is an exploded view of the CPAP device of FIG. 4.

### DETAILED DESCRIPTION OF THE PREFERRED EMBODIMENTS

Briefly, the present invention provides a CPAP device that includes the feature of having a blower mounted directly to the mouthpiece and/or nose mask that interfaces with the user. By this design, the CPAP device of the present invention does not require an air tube that conveys the pressurized air from the blower to the user's mouth and/or nose, as is typically found in conventional CPAP devices. Because the pressurized air has significantly less distance to travel from the blower to the user, air quality parameters such as air pressure, temperature, and relative humidity can be regulated more precisely. The improvement in the regulation and control of the relative humidity of the pressurized air is especially pronounced as a result of the reduced distance of air traversal. Data from a sleep study using a CPAP device according to a preferred embodiment of the present invention indicates that the absolute total air pressure needed for effective treatment of the disordered breathing events may be significantly reduced from average levels titrated on conventional CPAP devices, due to the fact that the dynamic volume of closed-loop area of pressure delivered to the airway is closer to the airway. Accordingly, the design of the present invention provides several advantages, including improved control of several air quality parameters, such as air pressure, temperature, and relative humidity; a more compact overall design; and a resultant improvement in user comfort. Further, the design of the present invention provides the unexpectedly positive result that, based on data from the clinical sleep study referred to above, patient compliance (i.e., consistency in wearing the CPAP device throughout an entire night's sleep) has been improved by using a CPAP device according to a preferred embodiment of the present invention, as compared with patient compliance using a conventional CPAP device.

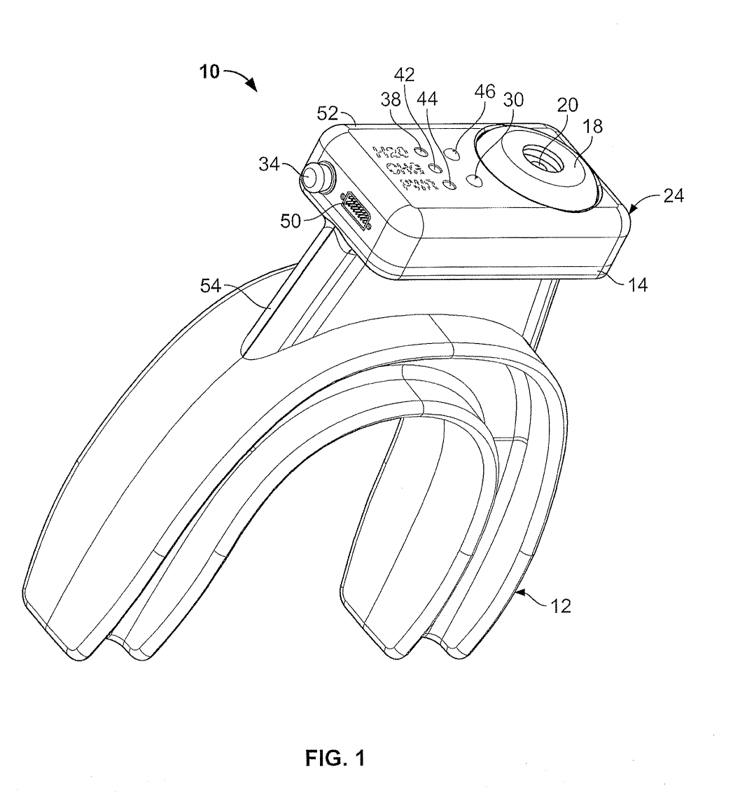

Referring now to FIGS. 1, 2, and 3, a preferred embodiment of the present invention is shown and generally designated by the reference numeral 10. The CPAP device 10 includes a mouthpiece 12, a housing 14, an airway 16, a blower 18, a cover 24, a power supply unit 26, a control circuit 28, and an on/off switch 30. The housing 14 is attached to the mouthpiece 12. The airway 16 is attached to the mouthpiece 12 and to the housing 14, and is configured to allow air to traverse through the mouthpiece 12 and the housing 14. The blower 18 is attached to the housing 14, and includes an inlet 20 and an outlet 22. The inlet 20 of the blower 18 is configured to be in fluid communication with the outlet 22. Further, the inlet 20 and the outlet 22 are each configured to be in fluid communication with the airway 16. The cover 24 is attached to the housing 14. The power supply unit 26 is attached to the housing 14 and operatively coupled to the blower 18. The control circuit 28 is attached to the housing 14 and operatively coupled to the power supply unit 26 and to the blower 18. The on/off switch 30 is attached to the cover 24 and operatively coupled to the control circuit 28. When the on/off switch 30 is in the on position, the blower 18 is configured to provide air at a positive pressure toward the mouthpiece and then into the user's mouth for the purpose of preventing the user from stoppages in respiration.

An optional humidifier chamber 32 and a cartridge 34 may be added to the device 10. The optional humidifier chamber 32 is attached to the housing 14 and to the blower 18, and is configured to be in fluid communication with the airway 16 and the outlet 22 of the blower 18. The optional cartridge 34 is attached to the humidifier chamber 32, and is configured to be in fluid communication with the airway 16. An optional air filter 36 may also be added to the device 10. The air filter 36 is attached to the blower 18 and is configured to be in fluid communication with the airway 16.

An optional light emitting diode (LED) 38 may be added to the device 10. The LED 38 is attached to the cover 24 and operatively coupled to the control circuit 28. The LED 38 maybe operatively coupled to the control circuit 28 for indicating conditions such as, for example, power charging conditions, on/off power conditions, and moisture conditions.

One or more optional detectors 40 may be added to the device 10. Each detector 40 is attached to the airway 16 and operatively coupled to the control circuit 28. The detector 40 may include one or more of a humidity detector, a temperature detector, a pressure detector, and an air flow detector. For example, a detector 40 may comprise a light-emitting diode that projects a beam through the mouthpiece 12 to the oral airway tissue; then, by light absorption detection, determine oxygen concentration of the air, similarly as a conventional Pluse oximeter. Alternatively, a detector 40 may comprise a sensor mounted on the mouthpiece 12 to record the heart rate. A plurality of detectors 40 may be used to enable a physician to conduct a polysomnography (i.e., a sleep study or sleep test), thereby collecting relevant data to enable the physician to diagnose sleep apnea. The relevant data may include heart rate, oxygen saturation, inspiratory and expiratory air flow, diaphragm effort level, and snoring data. Further, once a patient has been diagnosed, the efficacy of the CPAP device and the regulation of its settings may be measured on a nightly basis using the data obtained by the plurality of detectors 40.

An optional speaker 42 may be added to the device 10. The speaker 42 is attached to the cover 24 and operatively coupled to the control circuit 28. The speaker 42 may be used to communicate speakable items that are reflective of measured values, such as, for example, if expiration exceeds a normal value, then the user can be alerted to check the device. An optional microphone 44 may also be added to the device 10. The microphone 44 is attached to the cover 24 and operatively coupled to the control circuit 28. The microphone 44 may be used, for example, to record snoring. An optional combination of a speaker 42 and a microphone 44 may be added to the device 10. The control circuit 28 may be configured to receive an electrical input from the microphone 44 and to transmit an output to the speaker 42 to drive the speaker 42 to produce a noise cancellation function in response to snoring noises. Alternatively, the control circuit 28 may be configured to receive an electrical input from the microphone 44 and to transmit an output to the speaker 42 to drive the speaker 42 to produce siren alarm sounds in response to snoring noises. In another alternative, the control circuit 28 may be configured to receive an electrical input from the microphone 44 and to transmit an output to the speaker 42 to drive the speaker 42 to produce a musical melody in response to snoring noises.

An optional electrical socket 46 may be added to the device 10. The electrical socket 46 is attached to the cover 24 and operatively coupled to the control circuit 28. The electrical socket 46 is preferably used for charging the power unit 26.

An optional flash memory 48 may be added to the device 10. The flash memory 48 is attached to the housing 14 and operatively coupled to the control circuit 28. An optional parallel interface 50 may also be added to the device 10. The parallel interface 50 is attached to the cover 24 and operatively coupled to the control circuit 28. The parallel interface 50 may be used to download data stored in the flash memory 48 to an external device, such as a computer, or to recharge the power unit 26 from an external power source, such as a battery.

An optional heat pump 52 may be added to the device 10. The electrical socket 46 is attached to the cover 24 and operatively coupled to the control circuit 28. The heat pump 52 may be used in conjunction with a relative humidity sensor 40 to control the temperature and relative humidity of the pressurized air. The heat pump 52 may be any commercially available heat pump, such as, for example, a Peltier heat pump. An optional exhaust valve 54 may also be added to the device 10. The exhaust valve 54 is attached to the housing 14 and configured to be in fluid communication with the airway 16. The exhaust valve 54 is used to relieve any accumulated air pressure.

Referring to FIG. 1, a perspective view of a CPAP device 10 according to a preferred embodiment of the present invention is shown. The CPAP device 10 includes a mouthpiece 12, a housing 14, a blower 18, a cover 24, an on/off switch 30, a cartridge 34, an air filter 36, an LED 38, a speaker 42, a microphone 44, an electrical socket 46, a parallel interface 50, a heat pump 52, and an exhaust valve 54. The housing 14 is attached to the mouthpiece 12. The blower 18 is attached to the housing 14 and includes an inlet 20. The cover 24 is attached to the housing 14. The on/off switch 30 is attached to the cover 24. The cartridge 34 is mounted on the cover 24. The LED 38 is attached to the cover 24. The speaker 42 and the microphone 44 are each attached to the cover 24. The electrical socket 46, the parallel interface 50, and the heat pump 52 are each attached to the cover 24. The exhaust valve 54 is attached to the housing 14.

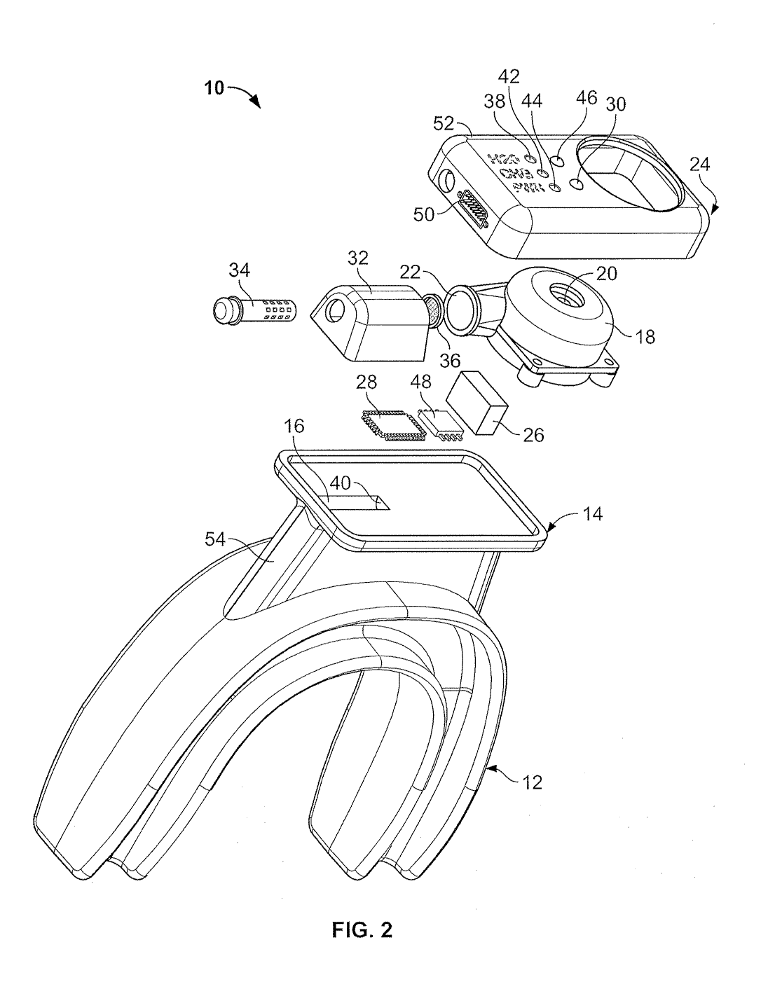

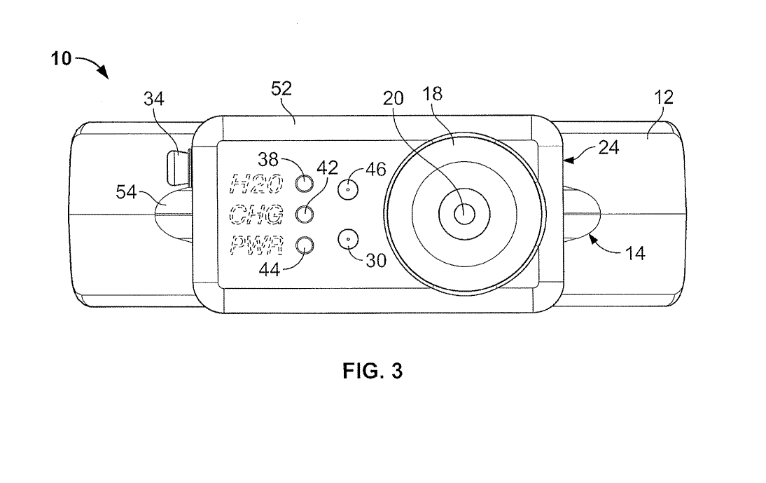

Referring to FIG. 2, an exploded perspective view of the CPAP device 10 of FIG. 1 is shown. In this exploded view, the airway 16 is illustrated within the housing 14, and is configured to enable air to traverse through the mouthpiece 12 and the housing 14. The detector 40 is positioned within the airway 16 to enable detection of one or more data relating to the air being breathed by the user. The blower 18 includes both an inlet 20 and an outlet 22. The flash memory 48 is also illustrated. The exhaust valve 54 is shown as being in fluid communication with the airway 16. Referring to FIG. 3, a frontal view of the CPAP device 10 of FIG. 1 is shown as being fully assembled.

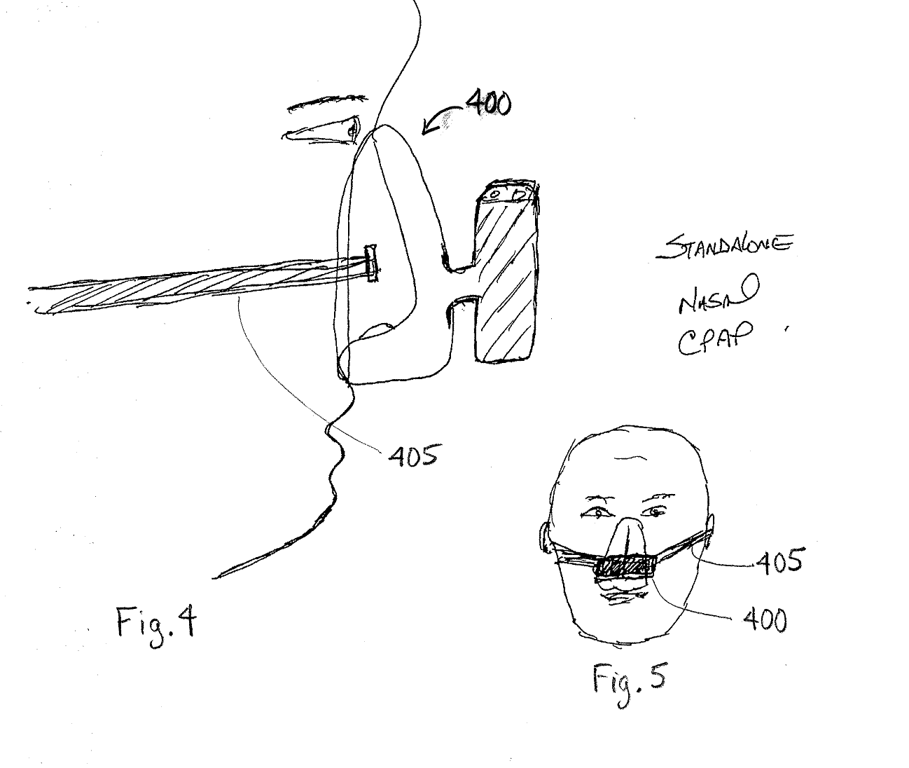

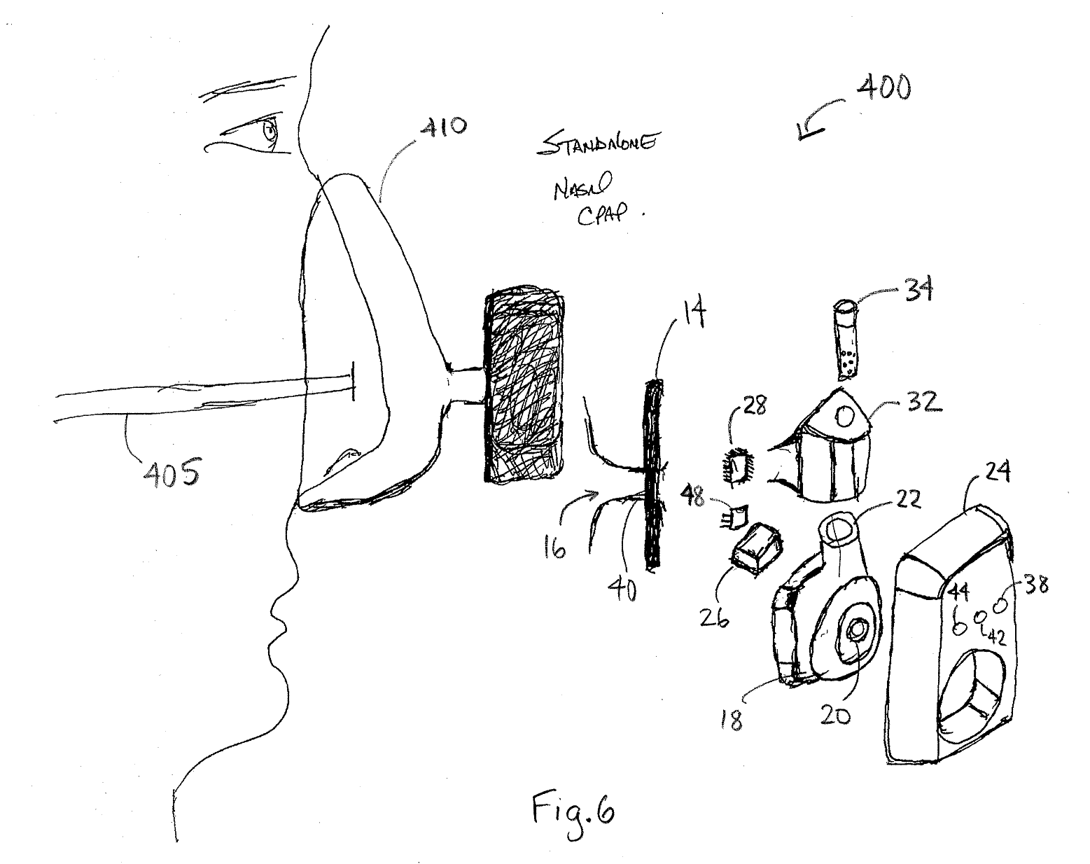

Referring to FIGS. 4, 5, and 6, in a second preferred embodiment, a CPAP device 400 having a nose mask that is configured to provide air directly into the nasal cavity is illustrated. In this embodiment, the CPAP device 400 does not include a mouthpiece; instead, the CPAP device 400 is designed to be worn by a user in a manner to completely and sealingly cover the user's nose, as illustrated in the side view of FIG. 4 and the frontal view of FIG. 5. Instead of the user biting down on a mouthpiece, in the present embodiment, a strap 405 is used to hold the CPAP device in place and to seal the nose mask while the user is sleeping.

Referring to FIG. 6, in the second preferred embodiment, the CPAP device 400 includes a nose mask 410 that fits over the user's nose. However, other than the nose mask 410, the device 400 includes many of the same components as described previously with respect to the first preferred embodiment. In particular, the CPAP device 400 also includes a housing 14, with an airway 16 and a detector 40; a control circuit 28; a flash memory 48; a power unit 26; a blower 18, with an inlet 20 and an outlet 22; a humidifier chamber 32; a cartridge 34; and a cover 24, which includes a speaker 42, a microphone 44, and an LED 38.

In a preferred embodiment, the control circuit 28 is a system on a chip (SOC). The control circuit 28 may include a 40 mm×25 mm×4 mm SOC that is powered by a rechargeable layered lithium strip power source that generates two independent DC 12-volt/1.3-watt power sources. One of the power sources is used to provide power to the blower 18, and the other power source is available to provide power to all of the other integrated circuit components. In addition to power management, the SOC provides several functions to the CPAP device 10, including: air pressure regulation; air flow regulation; relative humidity regulation; collection of air quality data; and integration of audio capabilities.

Regarding air pressure, the pressure setting is externally titrated, and the pressure setting is controlled by a digital pressure gauge, preferably in units of mm/Hg. Inspirations are reflected by negative pressure breathing, and expirations are reflected in positive pressure swings. Air flow by volume is controlled by an integrated thermocouple, which also indirectly represents airway temperature. Inhalations generally include cooler air, whereas exhalations generally include warmer air due to the internal body temperature. Regarding relative humidity, a small flow-through filter is used to calculate the relative humidity of the air passing through the CPAP device. Adjustment of the moisture concentration is achieved by a controlled release of water from a cylinder to maintain a predetermined target relative humidity. Regarding collection of air quality data, the SOC collects measurements of air pressure, inspiratory and expiratory flow durations, air temperature, and relative humidity of the air. Regarding integration of audio capabilities, the SOC is programmed to provide warning alerts when there are deviations or abnormalities in the collected air quality data, as well as an alarm-clock setting for waking the user (e.g., for medication), and audio tracks for assisting the user with sleep onset.

The air quality data collected by the SOC is typically stored in the flash memory 48. Accordingly, the flash memory 48 stores data relating to air pressure, inspiratory and expiratory flow durations, air temperature, and relative humidity. In addition, the flash memory 48 may include data relating to oxygen saturation levels, heat rate, snoring, and time of use. This data is useful for determining efficacy of the CPAP device and use of the treatment modality. The data stored on the flash memory 48 may be extracted from the device through the use of the parallel interface 50. The parallel interface 50 may also be used to recharge the power unit 26 by connection to an external battery or other power source.

According to either of the first and second preferred embodiments of the present invention, the CPAP device is intended to be used in one of at least three distinct modalities: 1) continuous air pressure; 2) bi-phase air pressure; and 3) automatic air pressure. The first of these modalities is continuous air pressure. In this first modality, positive air pressure is delivered into the airway (either nose, mouth, or both) at a titrated value continuously throughout the inspiratory/expiratory breathing phase of the user. A typical person is a negative pressure breather, with an atmospheric pressure of approximately 742 mm Hg in the lungs. As inspiration begins, a negative pressure gradient tends to reduce the pressure in the lungs from 742 mm Hg to approximately 738 mm Hg, in a similar manner as using a straw in a glass of water. In the situation where a person has sleep apnea, the negative pressure exceeds the airway wall retention threshold pressure, thus causing the wall of the airway to collapse and occlude the airway. This airway occlusion is defined as being an apneic event, and its duration is defined as being the time during which the airway is closed and no air is flowing through the airway.

By delivering continuous positive air pressure, the negative inspirations are shortened so as not to exceed the airway wall retention threshold pressure. The pressure needed to maintain the wall retention translates into a titrated pressure setting for the CPAP device. For a typical user, the titrated pressure setting usually falls within the range from 5.0 cm H₂O to 20.0 cm H₂O.

The second modality is bi-phase air pressure. In this second modality, the inspiratory pressure is the same as the continuous pressure used in the first modality. However, because exhalation is often difficult with a continuous positive air pressure in the airway, a bi-phase setting will provide a lower expiratory pressure to allow for easier expiration. For example, when using a bi-phase air pressure modality, the inspiratory pressure may be set to 8.0 cm H₂O and the expiratory pressure may be set to 5.0 cm H₂O. These titrated values are typically determined on a user-by-user basis.

The third modality is automatic air pressure. In this third modality, a sensor in the mouthpiece or nosepiece is used to control the air flow rate such that the air pressure is maximized at the lungs at all times. Accordingly, the air pressure being provided by the CPAP device is varying as a function of time, instead of being at either one or two constant predetermined values, as in the other two modalities.

In an alternative embodiment of the invention, a CPAP device may be configured to treat an airway closure using light therapy at selected wavelengths. It has been found that infrared light at a wavelength of approximately 680 nm can have an anti-inflammatory effect on human tissue, and further, that visible blue light at a wavelength of approximately 450 nm can increase intracellular communication. Therefore, by emitting light at selected wavelengths with a periodicity that is calculated in accordance with a user's inspiratory and expiratory intervals, it is possible to use a CPAP device to treat an airway closure using light therapy in lieu of continuous air pressure.

Referring to Tables 1 and 2, a sleep study was performed using a CPAP device according to a preferred embodiment of the present invention. As a control, the study also included data involving the same five subjects using a conventional CPAP device. In Tables 1 and 2, TIB stands for time in bed; TST stands for total sleep time; EFF % is TST divided by TIB; Oxyhemoglobin Saturation is a measure of oxygen saturation; REM stands for rapid eye movement sleep; and NREM stands for non-rapid eye movement sleep. Table 1 includes the data relating to the conventional CPAP device, and Table 2 includes the data relating to the CPAP device according to a preferred embodiment of the present invention.

TABLE 1

| Conventional | TIB | TST | | Pressure | Oxyhemoglobin | | | |
|---|---|---|---|---|---|---|---|---|
| CPAP Device | (min) | (min) | EFF % | (cm) | Saturation | Snoring | % REM | % NREM |
| Subject 1 | 191.5 | 142.0 | 74.2 | 8.0 | 96-88 | Occasional/ | 8 | 92 |
| | | | | | | Inspiratory | | |
| Subject 2 | 162.0 | 113.5 | 70.1 | 12.0 | 98-90 | None | 13 | 87 |
| Subject 3 | 184.0 | 152.0 | 82.6 | 7.5 | 96-91 | None | 14.5 | 85.5 |
| Subject 4 | 172.0 | 104.0 | 60.5 | 10.0 | 93-83 | Moderate/ | 9.5 | 90.5 |
| | | | | | | Inspiratory | | |
| Subject 5 | 193.0 | 162.0 | 83.9 | 7.0 | 95-89 | None | 15.5 | 84.5 |

TABLE 2

| New Invention | TIB | TST | | Pressure | Oxyhemoglobin | | | |
|---|---|---|---|---|---|---|---|---|
| CPAP Device | (min) | (min) | EFF % | (cm) | Saturation | Snoring | % REM | % NREM |
| Subject 1 | 180.0 | 149.5 | 83.1 | 8.0 | 97-93 | None | 11.5 | 88.5 |
| Subject 2 | 176.5 | 160.5 | 91.1 | 12.0 | 98-90 | None | 9.5 | 90.5 |
| Subject 3 | 181.0 | 162.0 | 89.5 | 7.5 | 97-91 | None | 10.0 | 90.0 |
| Subject 4 | 203.0 | 182.0 | 89.6 | 10.0 | 95-92 | None | 15.0 | 85.0 |
| Subject 5 | 184.0 | 163.5 | 88.9 | 7.0 | 96-92 | None | 8.5 | 91.5 |

As can be seen from the data in Tables 1 and 2, the effectiveness of the CPAP device according to a preferred embodiment of the present invention, as measured by total sleep time as a percentage of time in bed, is significantly increased for all five subjects as compared with the effectiveness of the conventional CPAP device. Accordingly, this data illustrates the improvements and advantages of the present invention as described above.

In another preferred embodiment of the present invention, a method of using a CPAP device comprises the steps of: providing a CPAP device as described above; mounting the CPAP device in a mouth of a user; activating the control circuit by using the on/off switch; detaching the cartridge from the humidifier chamber; moisturizing the detached cartridge; attaching the moisturized cartridge to the humidifier chamber; enabling the user to sleep while the device is mounted in the mouth of the user; collecting sleep response data via the control circuit; and storing the collected sleep response data in the flash memory. The method may further include the steps of coupling the parallel interface to a computer and downloading the stored sleep response data to the computer for analysis.

Numerous objects, features, and advantages of the present invention will be readily apparent to those of ordinary skill in the art upon study of this detailed description of exemplary embodiments of the present invention when taken in conjunction with the accompanying drawings. In this respect, it is to be understood that the present invention is not limited in its application to the details of construction and arrangement of components set forth herein. To the contrary, the invention is capable of other embodiments and of being practiced and carried out in various ways. Also, it is to be understood that the phraseology and terminology employed herein are for the purpose of description and should not be regarded as limiting. As such, those skilled in the art will appreciate that the present invention may readily be utilized as a basis for the design of other structures, methods, and systems for carrying out the several purposes of the present invention. It is important, therefore, that the claims be regarded as including such equivalent constructions insofar as they do not depart from the spirit and scope of the present invention.

While the present invention has been described with respect to what is presently considered to be the preferred embodiment, it is to be understood that the invention is not limited to the disclosed embodiments. To the contrary, the invention is intended to cover various modifications and equivalent arrangements included within the spirit and scope of the appended claims, including, for example, variations in size, materials, shape, form, function, and manner of operation, assembly, and use. The scope of the following claims is to be accorded the broadest interpretation so as to encompass all such modifications and equivalent structures and functions.

What is claimed is:

1. A continuous positive airway pressure (CPAP) system, comprising:
a mouthpiece configured for being positioned partially within a mouth of a user;
a housing mounted to the mouthpiece, the housing including an airway; and
a blower coupled directly to the housing, the blower being in fluid communication with the airway, wherein the blower is configured to provide pressurized air to the mouth of the user for preventing respiration stoppages.

2. The CPAP system of claim 1, further comprising a humidifier chamber coupled directly to the blower and to the housing, the humidifier chamber being in fluid communication with the airway, and a cartridge coupled directly to the humidifier chamber.

3. The CPAP system of claim 2, further comprising a control circuit, the control circuit being operatively coupled to the blower, and at least one detector, the at least one detector being operatively coupled to the control circuit, wherein the control circuit is configured to control a mode of operation selected from the group consisting of a continuous mode, a bi-phase mode, and an automatic mode.

4. The CPAP system of claim 3, wherein the at least one detector comprises an air pressure detector, and wherein the control circuit is further configured to control a pressure level of the pressurized air.

5. The CPAP system of claim 3, wherein the at least one detector comprises a temperature detector, and the control circuit is further configured to control a temperature of the pressurized air.

6. The CPAP system of claim 3, wherein the at least one detector comprises a humidity detector, and the control circuit is further configured to control a relative humidity of the pressurized air.

7. The CPAP system of claim 3, further comprising a flash memory, the flash memory being operatively coupled to the control circuit and the at least one detector, and the flash memory being configured to store data detected by the at least one detector.

8. The CPAP system of claim 3, further comprising a microphone, the microphone being operatively coupled to the control circuit, and a speaker, the speaker being operatively coupled to the control circuit, wherein the microphone is configured to detect snoring noises emitted by the user, and the speaker is configured to output audible sound.

9. The CPAP system of claim 8, wherein the audible sound is selected from the group consisting of a noise cancellation function with respect to the detected snoring noises, an alarm sound for awakening the user from sleeping, and a musical melody.

10. A continuous positive airway pressure (CPAP) system, comprising:
a nose mask configured for being sealingly positioned over a nose of a user;
a housing mounted to the nose mask, the housing including an airway; and
a blower coupled directly to the housing, the blower being in fluid communication with the airway, wherein the blower is configured to provide pressurized air to a nasal cavity of the user for preventing respiration stoppages.

11. The CPAP system of claim 10, further comprising a humidifier chamber coupled directly to the blower and to the housing, the humidifier chamber being in fluid communication with the airway, and a cartridge coupled directly to the humidifier chamber.

12. The CPAP system of claim 11, further comprising a control circuit, the control circuit being operatively coupled to the blower, and at least one detector, the at least one detector being operatively coupled to the control circuit, wherein the control circuit is configured to control a mode of operation selected from the group consisting of a continuous mode, a bi-phase mode, and an automatic mode.

13. The CPAP system of claim 12, wherein the at least one detector is selected from the group consisting of an air pressure detector, a temperature detector, and a humidity detector.

14. The CPAP system of claim 12, further comprising a flash memory, the flash memory being operatively coupled to the control circuit and the at least one detector, and the flash memory being configured to store data detected by the at least one detector.

15. The CPAP system of claim 12, further comprising a microphone, the microphone being operatively coupled to the control circuit, and a speaker, the speaker being operatively coupled to the control circuit, wherein the microphone is configured to detect snoring noises emitted by the user, and the speaker is configured to output audible sound, the audible sound being selected from the group consisting of a noise cancellation function with respect to the detected snoring noises, an alarm sound for awakening the user from sleeping, and a musical melody.

16. A method for treatment of sleep apnea in a user, the method comprising the step of providing pressurized air to a mouth of the user by using a continuous positive airway pressure (CPAP) device,
wherein the CPAP device includes a mouthpiece configured for being positioned partially within the mouth of the user, a housing mounted to the mouthpiece and having an airway, and a blower coupled directly to the housing and in fluid communication with the airway.

17. The method of claim 16, the method further comprising the steps of:
selecting a mode of operation of the CPAP device from the group consisting of a continuous mode, a bi-phase mode, and an automatic mode, and
regulating a pressure of the pressurized air based on the selected mode of operation.

18. The method of claim 16, the method further comprising the step of regulating a relative humidity of the pressurized air.

19. The method of claim 16, the method further comprising the step of regulating a temperature of the pressurized air.

20. The method of claim 16, the method further comprising the steps of detecting data relating to respiration by the user and recording the detected data.

21. The method of claim 20, wherein the step of detecting data relating to respiration comprises detecting snoring noises emitted by the user.

22. The method of claim 21, the method further comprising the step of outputting audible sound, the audible sound being selected from the group consisting of a noise cancellation function in respect of the detected snoring noises, an alarm sound for awakening the user from sleeping, and a musical melody.

23. A method for treatment of sleep apnea in a user, the method comprising the step of providing pressurized air to a nasal cavity of the user by using a continuous positive airway pressure (CPAP) device,
wherein the CPAP device includes a nose mask configured for being sealingly positioned over a nose of the user, a housing mounted to the nose mask and having an airway, and a blower coupled directly to the housing and in fluid communication with the airway.

24. The method of claim 23, the method further comprising the steps of:
selecting a mode of operation of the CPAP device from the group consisting of a continuous mode, a bi-phase mode, and an automatic mode, and
regulating a pressure of the pressurized air based on the selected mode of operation.

25. The method of claim 24, the method further comprising the step of regulating a predetermined quality of the pressurized air, the predetermined quality being selected from the group consisting of temperature and relative humidity.
